(19) United States
(12) Patent Application Publication
(10) Pub. No.: US 2006/0224079 A1
(43) Pub. Date: Oct. 5, 2006

(54) Edema monitoring system and method utilizing an implantable medical device

(76) Inventor: Bohdan O. Washchuk, Minneapolis, MN (US)

Correspondence Address: MEDTRONIC, INC., 710 MEDTRONIC PARK, MINNEAPOLIS, MN, 55432-9924, US

(21) Appl. No.: 11/096,945

(22) Filed: Mar. 31, 2005

(51) Int. Cl. A61B 5/05

(52) U.S. Cl. 600/547

(57) ABSTRACT

An edema monitoring system includes an implantable medical device (IMD) and a personal edema monitor. The IMD measures an intra-thoracic impedance and transmits intra-thoracic impedance data and other biological data to the personal edema monitor, which generates a user interface based on patient inputs relating to activities and health assessments, the measured intra-thoracic impedance data and the other biological data.

### BACKGROUND OF THE INVENTION

The present invention relates to a system and method for edema monitoring utilizing an implantable medical device (IMD).

Heart failure afflicts 5 million Americans and is the number one cause of hospital admissions today. Most of these hospital admissions are the result of fluid accumulation in the thorax, which often goes undetected until a patient is critically ill. It is not unusual for patients to require hospitalization or urgent treatment at an emergency room for severe respiratory distress. With approximately 1 million hospitalizations each year for heart failure, heart failure management is a tremendous cost burden to the healthcare system.

Various methods have been devised to monitor the accumulation of fluid in the thorax, also known as edema. One method is to have the patient weigh himself each day, or multiple times per day, and monitor for sudden weight changes. If the patient notices two or three pounds of weight gain per day over a period of a few days, the patient is instructed to notify a physician. Unfortunately, this method is imprecise and prone to error. It is difficult to know whether weight gain is due to an improvement in the patient's health resulting in increased eating or muscle gain, or whether the weight gain is due to fluid accumulation. As a result, a buildup of fluid can remain undetected or misdiagnosed.

IMDs are now capable of measuring intra-thoracic impedance (a measure of the impedance within a portion of the thorax), which is inversely correlated to the amount of fluid in the thorax. Generally, as the amount of fluid in the thorax increases, the intra-thoracic impedance decreases.

Current IMDs are capable of communicating a measured intra-thoracic impedance value to a monitoring system used by a care giver. However, these systems require that the care giver regularly check the measured values and compare them with previous values, which imposes an undesirable burden on the care giver. In addition, the care giver is generally unaware of what the patient may have done to influence the impedance values, and as a result, has little context for evaluation of those values.

Interaction with a patient has been in the form of emergency alerts that notify the patient with an alarm that a threshold value of intra-thoracic impedance has been exceeded and that urgent action is needed. Any further interaction with the patient has been limited to that provided by the care giver directly. There is a need for an edema monitoring system and method utilizing an IMD, which enables the patient to be involved in the monitoring and treatment of his or her own condition.

### BRIEF SUMMARY OF THE INVENTION

An edema monitoring system includes an implantable medical device and a personal edema monitor. The implantable medical device measures an intra-thoracic impedance and transmits the intra-thoracic impedance to the personal edema monitor, which generates a user interface to provide a representation related to the measured intra-thoracic impedance to the patient.

### BRIEF DESCRIPTION OF THE DRAWINGS

FIG. 1 illustrates an edema monitoring system of the present invention including an implantable medical device and a personal edema monitor.

FIG. 2 is a block diagram of the implantable medical device.

FIG. 3 is a block diagram of the personal edema monitor.

FIG. 4 illustrates various formats of the user interface displayed by the personal edema monitor.

FIG. 5 illustrates another screen of the user interface displayed by the personal edema monitor.

### DETAILED DESCRIPTION

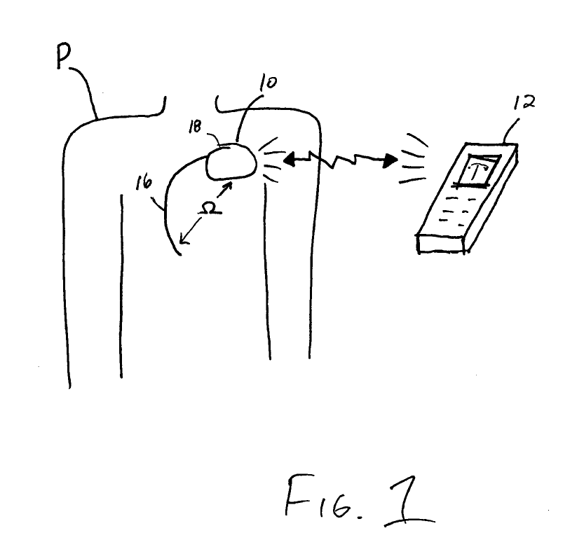

FIG. 1 illustrates the impedance monitoring system of the present invention, which includes IMD 10 and personal edema monitor (PEM) 12. IMD 10 is, for example, an implantable cardioverter-defibrillator (ICD) or an implantable pulse generator (IPG). IMD 10 is capable of measuring the intra-thoracic impedance (□) within patient P, storing the impedance in memory, and transmitting the impedance and other related data to PEM 12.

PEM 12 receives the measured impedance data and generates a user interface that provides a user friendly interpretation of the impedance data to the patient. In addition, PEM 12 can prompt the user to supply additional information to assist PEM 12 in the interpretation of the impedance data. PEM 12 can take the form of a personal digital assistant (PDA), a handheld computer, a tablet PC, or other special or general purpose device capable of receiving and displaying information from IMD 10.

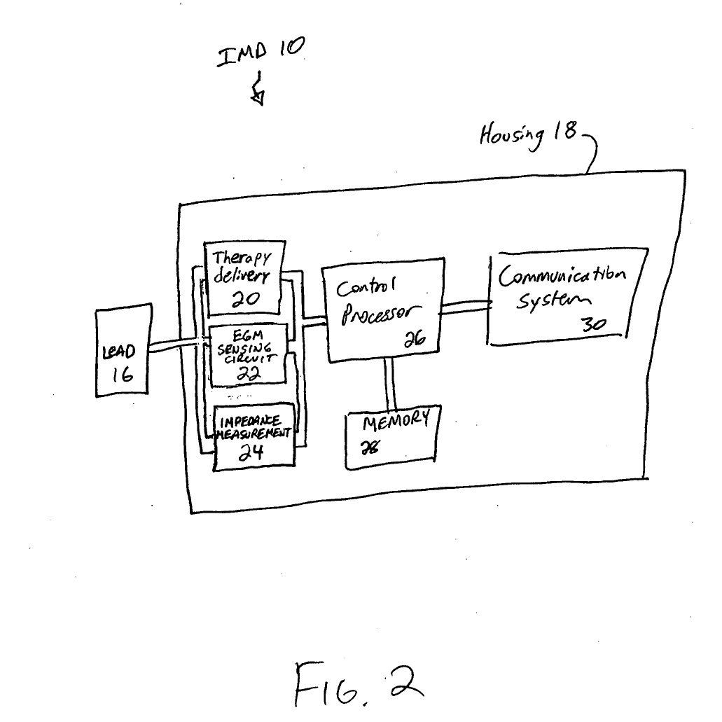

Referring to FIG. 2, IMD 10 measures the intra-thoracic impedance by sending an electrical pulse from a lead 20 into the thoracic cavity of patient P. The pulse travels through a portion of the thoracic cavity to housing 18 of the IMD. IMD 10 calculates the impedance of the thoracic cavity and stores this value in memory. After one or more impedance measurements have been stored, communication is initiated to transmit the stored data to PEM 12. An output based on the data is then displayed on PEM 12 to inform the patient of the impedance of the thoracic cavity, rates of change of impedance, relative wetness or dryness (measures of hypervolemia, hypovolemia, and euvolemia), or other relevant information. In this way, patient P is provided with the opportunity to monitor his own condition and evaluate the results. If the results indicate a trend of changing intra-thoracic impedance, patient P takes the appropriate action such as contacting a care giver or taking medications as instructed by the caregiver.

FIG. 2 is a block diagram of IMD 10 including lead 16, housing 18, therapy delivery 20, electrogram (EGM) sensing circuit 22, impedance measurement circuitry 24, control processor 26, memory 28, and communication system 30. IMD 10 is, for example, an implantable cardioverter-defibrillator (ICD). Lead 16 extends from housing 18 into the heart and includes tip and ring electrodes for delivery of pacing pulses and a coil electrode for delivery of defibrillation therapies. Housing 18 provides a protective enclosure for IMD 10 and is electrically connected to the negative terminal of the battery so as to function as the electrical ground of IMD 10. Housing 18 is spaced from the electrodes of lead 16 across a portion of the thoracic cavity. Measurement of intra-thoracic impedance is performed between the coil electrode of lead 16 and housing 18.

Electrical signals are generated or detected on lead 16 by lead electronics including therapy delivery circuit 20, EGM sensing circuit 22, and impedance measurement circuit 24. Therapy delivery circuit 20 provides therapies including defibrillation shocks and pacing pulses. EGM sensing circuit 22 detects intrinsic cardiac signals from the heart, which are used to select and control the therapy delivered. Impedance measurement circuit 24 measures the voltage and current of an electrical pulse between the coil electrode of lead 16 and housing 18 for determination of the impedance of the intra-thoracic cavity.

Control processor 26 controls and monitors the operation of circuits 20, 22 and 24 and processes data which it stores in memory 28 and transmits through communication system 30. Communication system 30 is a bidirectional radio frequency (RF) communication system, although other forms of wireless communication can also be used.

When an intra-thoracic impedance measurement is desired to assess the amount of fluid in the thoracic cavity, control processor 26 instructs therapy delivery circuitry 20 to deliver a pulse to the coil electrode of lead 16. The pulse travels from therapy delivery circuit 20, through lead 16 to the coil electrode, and then through a portion of the intra-thoracic cavity to housing 18.

During the pulse delivery, control processor 26 instructs impedance measurement circuit 24 to perform a number of measurements. One of these measurements is the voltage of the pulse (measured as the voltage difference from lead 16 to housing 18). The other measurement is the voltage across a small internal resistor connected between the positive terminal of the battery and lead 16. Knowing the voltage across the internal resistor during the voltage pulse, the resistance of the resistor, and the voltage of the pulse, the impedance of the thoracic cavity can be calculated using Ohm's Law. Further detail regarding the measurement and calculation of intra-thoracic impedance with an implantable medical device can be found in U.S. Publication No. 2004/0172080, filed Oct. 23, 2002 for METHOD AND APPARATUS FOR DETECTING CHANGE IN INTRATHORACIC ELECTRICAL IMPEDANCE by R. Karschnia and M. Peluso.

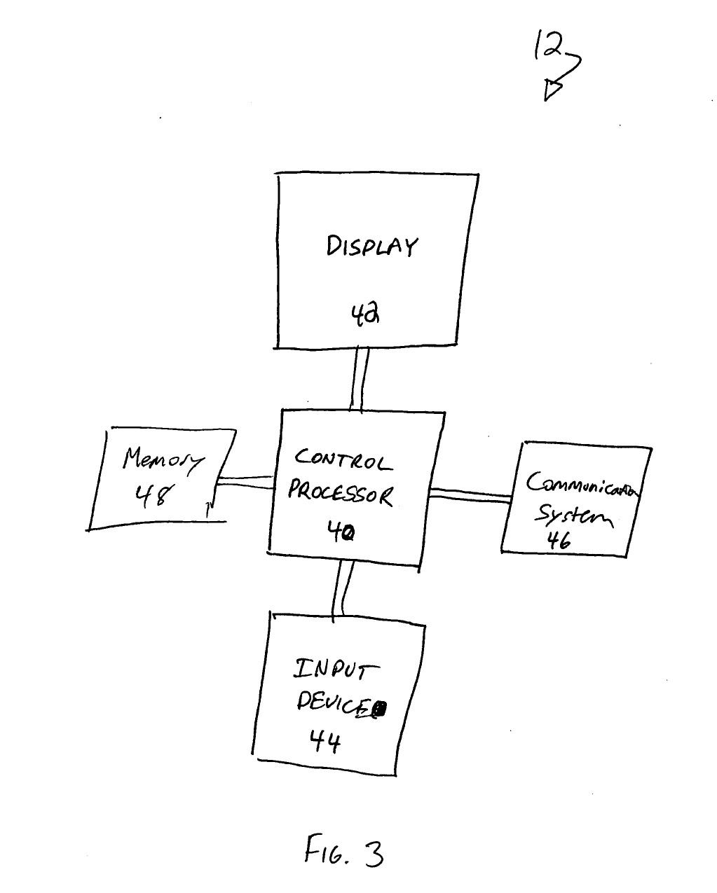

FIG. 3 is a block diagram of PEM 12, which includes control processor 40, display 42, input device 44, communication system 46, and memory 48. Control processor 40 performs calculations and controls the overall operation of PEM 12. Display 42 is a liquid-crystal display or other visual display capable of displaying numbers, symbols, graphs, charts, or other visual indicators. Input device 44 is a keyboard, button, stylus, touch screen or other input device for receiving input from patient P. Communication system 46 is a telemetry system capable of bi-directional communication with IMD 10. Memory 48 stores programs for use by processor 40, as well as data received from IMD 10.

PEM 12 enables patient P to monitor his own condition by displaying information based on intra-thoracic impedance measurements in a user-friendly format. PEM 12 receives the impedance measurement data transmitted from IMD 10, stores the data in memory 48, processes the data, and displays a representation or interpretation of the data on display 42. Communication between PEM 12 and IMD 10 may be user-initiated or initiated automatically by PEM 12 or IMD 10 periodically, or when a threshold value has been exceeded. Furthermore, PEM 12 may include a patient alert feature to notify patient P that action is needed.

Input device 44 enables PEM 12 to receive input so that patient P can modify the display format of information and can provide additional data to assist in more accurate interpretation of measured impedance data. For example, activity or the consumption of dehydrating foods or beverages are both factors that can influence the intra-thoracic impedance. With PEM 12, this information can be entered, stored, and displayed to enable more accurate evaluation of the patient's current condition.

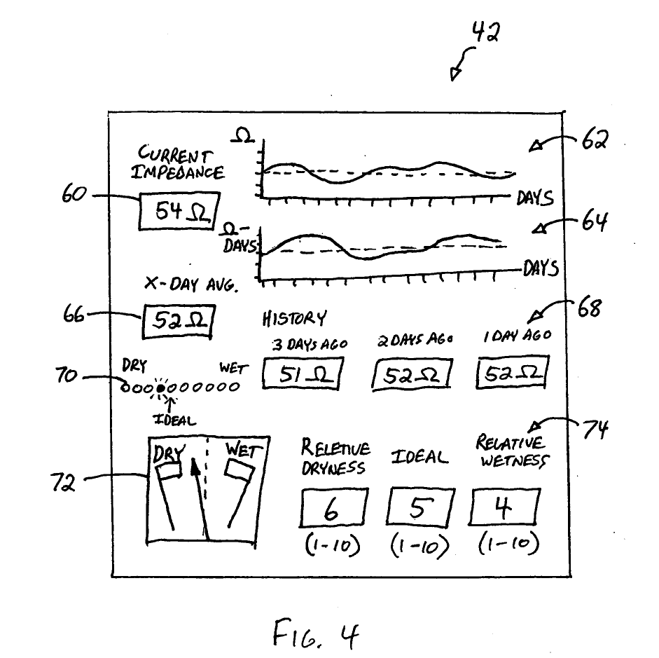

FIG. 4 illustrates various display formats for the intra-thoracic impedance data. The display formats inform patient P of the current impedance measurements, the wetness or dryness of the thoracic cavity, rates of change of impedance over time, and other related indications of fluid level or impedance. Not all of the displayed information shown in FIG. 4 need be presented at the same time. Furthermore, many other user-friendly display formats can also be used to convey the same or related information to patient P. PEM 12 can also be configured to wirelessly transmit the information to another device where it is stored, printed, or displayed.

In one embodiment, display formats shown on display 42 include current impedance indicator 60, impedance graph 62, integral difference graph 64, X-day average indicator 66, history indicator 68, dryness/wetness scale 70, dryness/wetness gauge 72, relative dryness/wetness indicators 74. Impedance indicator 60 displays the most current data from impedance measurements. Alternatively, impedance indicator 60 can provide an average of the most recent impedance measurements over a period of time. Providing an average rather than simply the last measurement yields a numerical output with less short-term variability and which is less effected by short-term factors.

This same data can also be displayed on impedance graph 62, which plots the impedance over a period of time. The graphical form is beneficial in showing trends, and also enables patient P to view and compare impedance changes with other factors. For example, if patient P exercised vigorously one day, patient P can see what effect the exercise had on the intra-thoracic impedance measurements during the period that followed.

PEM 12 also displays integral difference graph 64, which provides more information relating to changes in intra-thoracic impedance. Specifically, integral difference graph 64 plots the integral of the difference between the measured impedance and a baseline (or ideal) value, resulting in a display in units of □-days over a certain period of time. Integral difference graph 64 is useful in detecting a trend of small impedance changes over a period of time that indicates a gradual change in fluid level in the thoracic cavity.

X-day average 66 is calculated by PEM 12 by averaging the impedance measurements over a period of X days, where X is selected by either the patient or the care giver.

History display 68 provides a numerical indicator of the daily average impedance values for the past three days. This enables patient P to compare current impedance (60) to past impedances (68) and recognize changing trends in intra-thoracic impedance.

Some patients may find an impedance value display to be counter-intuitive since a decreased impedance corresponds to an increased amount of fluid in the thoracic cavity. In addition, some patients will be unfamiliar with the meaning of an impedance. To provide a more intuitive and easily understood display, indicators 70, 72, and 74 are provided that indicate the relative dryness or wetness of the thoracic cavity as compared to an ideal or baseline value.

Dryness/wetness scale 70 and dryness/wetness gauge 72 both indicate the current dryness or wetness of the thoracic cavity compared to the baseline value. Dryness/wetness scale 70 includes a row of light-emitting diodes, or graphical representations of LEDs. One of the LEDs is illuminated to indicate the relative dryness or wetness of the thoracic cavity compared to the baseline value. For example, if the intra-thoracic impedance is very low, the left-most LED is illuminated to show that the thoracic cavity is very dry. If the intra-thoracic impedance is very high, the right-most LED is illuminated to show that the thoracic cavity is very wet. Accordingly, LEDs between the left-most and right-most LEDs represent various degrees of dryness or wetness. Similarly, dryness/wetness gauge 72 indicates the relative dryness or wetness of the thoracic cavity with an arrow or cursor that points in a direction indicative of the relative wetness or dryness.

Relative numerical indicators 74 are numerical displays which indicate the current moisture content of the thoracic cavity by displaying a number from 1 to 10. Dryness indicator indicates the relative dryness where 1 is very wet and 10 is very dry. Similarly, wetness indicator 68 displays the relative wetness where 1 is very dry and 10 is very wet. Ideal indicator 66 displays the ideal dryness or wetness that is desired. This enables patient P to easily compare their present value with the desired value and take action accordingly.

Alternatively, only one of the dryness or wetness indicators is displayed along with the ideal indicator. For example, some patients may prefer to monitor relative dryness as opposed to relative wetness. This perspective emphasizes the positive, rather than emphasizing the negative, and encourages the patient to participate in monitoring the fluid condition.

All of the display formats shown in FIG. 4 provide a means for patient P to self-monitor the fluid level and intra-thoracic impedance over time. If sudden changes are noted over a period of days or weeks, action should be taken by patient P as specified by the care giver.

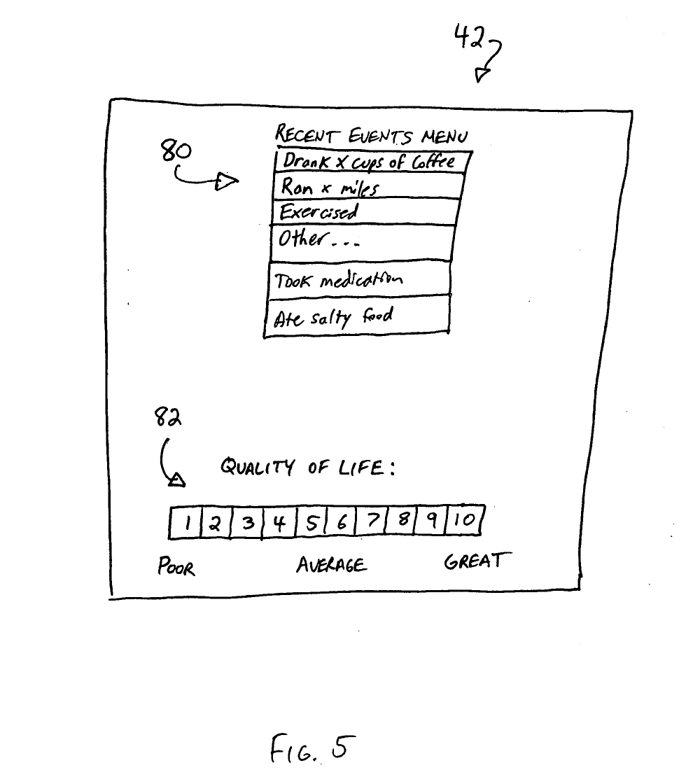

FIG. 5 illustrates another user interface screen on display 42 of PEM 12. This user interface screen enables patient P to provide input or feedback to PEM 12 relating to recent events and the patient's current quality of life. The information can be used by PEM 12 to more accurately assess the patient's condition, or to communicate to a care giver who reviews the data from IMD 10 and the inputs from patient P stored by PEM 12. Patient P enters feedback information using recent events menu 80 and quality of life menu 82.

Recent events menu 80 enables patient P to input information related to factors that influence intra-thoracic impedance, such as activity level, food or beverage consumption, and medications. For example, after patient P takes a prescribed medication, the “Took Medication” option is selected from the menu of recent events. This option can be programmed by the care giver to the normal dose of medication, or alternatively an additional screen is presented that prompts patient P to enter the amount of medication taken. Similarly, information about activity, consumption of drinks such as coffee or alcoholic beverages, consumption of salty foods, or any other relevant factors can be entered using recent events menu 80. This information can be used by PEM 12 to make adjustments to the displays shown in FIG. 4. In particular, the ideal or baseline values can be adjusted as necessary in response to the input information.

Quality of life menu 82 enables patient P to input a personal assessment. “Quality of life” is a general phrase indicating the overall feeling of health of the patient including energy level, ease of breathing, clarity of thought, and other factors relating to general health and wellness. Patient P enters his perception of his current quality of life by selecting a number from 1 to 10, where 1 represents a poor quality of life and 10 represents a great quality of life. PEM 12 stores this information and can use it to adjust the displays shown in FIG. 4.

One of the benefits of receiving a quality of life input from patient P is that it enables PEM 12 to more accurately determine the ideal intra-thoracic impedance for patient P. This is desirable because a particular intra-thoracic impedance value that is ideal for one patient may not be ideal for another patient. By enabling patient P to provide feedback to PEM 12 the patient's current quality of life can be compared to the most recent impedance measurements and the current ideal value, as well as to previous quality of life assessments. PEM 12 can use this information to select the ideal impedance for patient P. PEM 12 continues to fine-tune the ideal impedance over time as more feedback from patient P is received. As a result, PEM 12 assists patient P and the care giver in knowing the most desirable intra-thoracic impedance for that patient, and guides them in taking appropriate action. PEM 12 stores data from IMD 10 as well as inputs from patient P, so that the caregiver can review the information during patient visits or by a download from PEM 12 to the caregivers computer via the Internet.

PEM 12 can also use biological data such as EGM or other data from IMD 10 to more accurately interpret the patient's condition and evaluate potential causes of detected changes. For example, if PEM 12 receives information from IMD 10 that shows that an atrial tachycardia (AT) was detected at a particular time, that information can be compared with the changes in intra-thoracic impedance to evaluate whether AT is the cause of the impedance changes. Any other data stored in IMD 10 can also be used to assist in the interpretation and evaluation of the patient's condition, and adjust the displays accordingly, such as data relating to atrial fibrillation, ventricular tachycardia, ventricular fibrillation, heart rate variability, cardiac resynchronization therapy, pacing percentages, rate response information, frequency of episodes, and burden of episodes.

Although the present invention has been described with reference to preferred embodiments, workers skilled in the art will recognize that changes may be made in form and detail without departing from the spirit and scope of the invention. For example, it is recognized that impedance measurements can be taken between leads, rather than between a lead and the housing. It is also recognized that other means of communicating information to the patient may be used such as audible sounds (voices, tones, or other sounds) or vibrations (such as by pulsing a certain number of times indicative of the current condition).

1. An edema monitoring system comprising:
an implantable medical device to measure an intra-thoracic impedance and to transmit intra-thoracic impedance data; and
a personal edema monitor to receive the intra-thoracic impedance data and patient inputs and to generate a user interface based on the measured intra-thoracic impedance data and the patient inputs.

2. The system of claim 1, wherein the personal edema monitor is a hand-held device.

3. The system of claim 1, wherein the user interface displays a representation of patient health, based on the measured intra-thoracic impedance, to the patient.

4. The system of claim 1, wherein the user interface provides instructions for entry of patient inputs representing an assessment of patient quality of life.

5. The system of claim 1, wherein the user interface guides entry of patient inputs relating to activities that have an effect on edema.

6. A method of monitoring edema, the method comprising:
measuring an intra-thoracic impedance with an implantable medical device;
transmitting intra-thoracic impedance data from the implantable medical device to a personal edema monitor; and
generating an output based on the intra-thoracic impedance data on a user interface of the personal edema monitor.

7. The method of claim 6, wherein generating an output comprises displaying a numerical impedance.

8. The method of claim 6, wherein generating an output comprises displaying a visual representation of the relative dryness of the thoracic cavity.

9. The method of claim 6, wherein generating an output comprises displaying a visual representation of the relative wetness of the thoracic cavity.

10. The method of claim 6, wherein the output is related to an amount of fluid in a thoracic cavity.

11. The method of claim 6 and further comprising: receiving biological data from the implantable medical device; and
determining the output as a function of the intra-thoracic impedance data and the biological data.

12. The method of claim 6 and further comprising:
receiving patient input representing information affecting edema; and
determining the output as a function of the intra-thoracic impedance data and the patient input.

13. The method of claim 6, further comprising:
displaying selectable options on the user interface of the personal edema monitor;
receiving an input related to the selectable options; and
providing an output based on the intra-thoracic impedance data and the input.

14. The method of claim 13, wherein the input represents an assessment of quality of life.

15. The method of claim 13, wherein the input represents information regarding a recent event.

16. A personal edema monitor comprising:
means for receiving data from an IMD relating to an impedance measurement of an intra-thoracic cavity;
means for storing the data;
means for receiving inputs relating to patient health and activities; and
means for processing the data and generating a user interface that displays an output based on the data and the inputs.

17. The personal edema monitor of claim 16, wherein the means for receiving data is a bidirectional radio-frequency communication system.

18. The personal edema monitor of claim 16, wherein the inputs represent an assessment of quality of life.

19. The personal edema monitor of claim 16, wherein the inputs represent information regarding physical activity.

20. The personal edema monitor of claim 16, wherein the inputs represent an assessment of food and beverage intake.
